(19) United States
(12) Patent Application Publication
(10) Pub. No.: US 2007/0172434 A1
(43) Pub. Date: Jul. 26, 2007

(54) Antimicrobial dental materials, restorations, and prostheses

(76) Inventors: Gary R. Jernberg, North Mankato, MN (US); Richard J. Simonsen, Mesa, AZ (US)

Correspondence Address: MERCHANT & GOULD PC, P.O. BOX 2903, MINNEAPOLIS, MN, 55402-0903, US

(21) Appl. No.: 11/338,616

(22) Filed: Jan. 24, 2006

(51) Int. Cl. A61K 8/49; A61K 8/41

(52) U.S. Cl. 424/49; 977/906

(57) ABSTRACT

The present invention relates to compositions and methods for antimicrobial dental materials in the restoration of the sequelae of oral infections including replacement of oral tissues lost to disease and prevention of additional infections.

### TECHNICAL FIELD OF THE INVENTION

The present invention relates to compositions and methods for antimicrobial dental materials. These dental materials reduce the likelihood or severity of oral infections during restoration or replacement of oral tissues lost to disease. The antimicrobial dental materials include, but are not limited to, dental restorations, dental coatings and sealants, preventive varnishes, dental prosthetic restorations, crown and bridge prosthetics, removable partial and full dentures, dental cements and luting agents, dental bases and liners, dental (cavity-lining) varnishes, dental implants, fixed partial and full dentures, endodontic materials and orthodontic appliances, bands, brackets and space maintainers.

### BACKGROUND OF THE INVENTION

Oral diseases are a major affliction to mankind. Dental caries (tooth decay) and periodontal diseases are the major diseases affecting the oral cavity. Bacterial plaque is the principal causative agent of dental caries and periodontal disease.

Dental caries, or tooth decay, is a pathological process of localized destruction of tooth tissues by microorganisms (Latin: caries=rottenness). Dental caries is experienced by most Americans to some degree, mostly before reaching adulthood. In the past two decades there have been tremendous advances in the understanding of the multifactorial etiology of caries, of the specific flora associated with smooth surface, pit and fissure, and root caries, the transmissibility of the flora, and the formation and the mechanisms involved in the adherence of dental plaque. There is, now, a better appreciation for the unique role dietary sucrose plays in the etiology of dental caries. Caries continues to be a major public health problem despite many scientific advances. The repair or replacement of carious teeth involves millions of work-hours for those afflicted. Americans spend about $20 billion a year to treat all their dental problems. A major portion of this amount is for the treatment of carious teeth, or treatment of the resultant breakdown from carious infections. Dental caries is a multifactorial and complex disease involving the simultaneous interplay of three principal factors—the microflora, the host and the substrate (diet).

Fluoride therapy continues to be the cornerstone of any caries-preventive program. Fluoride-containing dentifrices, 0.76% sodium monofluorophosphate, or 0.22% sodium fluoride, account for over 90% of the market in the United States. Other self-application procedures for the delivery of topical fluorides involve prescription items and include mouth rinsing with fluoride solutions and applying fluoride gels in mouthpieces. With the combination of systematic and topical fluoride applications, the prevalence of smooth surface caries has greatly diminished over the past 50 years. Pits and fissures in the occlusal surfaces of permanent teeth are particularly susceptible to decay, and fluoride treatments have been least effective in preventing caries in these areas. The susceptibility of occlusal pits and fissures to caries is related to the physical character and morphology of the individual pit or fissure, which can provide shelter for organisms and obstruct oral hygiene procedures. Pits and fissures on the occlusal surfaces of posterior teeth are more susceptible to caries because the morphology of the surface structure is irregular and there is opportunity for food retention and bacterial proliferation leading to caries initiation.

These surfaces can be dealt with by applying an adhesive resin coating to obtund the irregularities and create a non-retentive smooth surface that is less likely to decay. The most common sealants are based on Bis-GMA resin and are light cured and activated by a diketone and an aliphatic amine. The first generation of chemically-initiated Bis-GMA sealants was polymerized by an organic amine accelerator; commercial self-cured sealants are still available. The material is supplied as a two-component system: one component contains Bis-GMA resin and benzoyl peroxide initiator, and the other contains Bis-GMA resin with 5% organic amine accelerator.

Low-viscosity, high-flow composites marketed as flowable composites are advocated for a wide variety of applications, such as resin restorations, cavity liners, restoration repairs, and cervical restorations. These applications are not well supported with data, but their clinical use is widespread. Flowable composites are usually packaged in syringes or in computes. These can be used for direct application to the cavity or the tooth surface. Because of their documented slow release of fluoride, glass ionomers are used in cervical and Class V restorations in adults where esthetics is not critical. They are specifically recommended for patients with high caries risk. Hybrid ionomers or resin-modified glass ionomers are used for restorations in low stress-bearing areas and are recommended for patients with high caries risk. These restorations are more esthetic than glass ionomers because of their resin content. See E. Newbrun, Cariology, Third Edition, Quintessence. See R. Craig, Restorative Dental Materials, Eleventh Edition, Elsevier.

Periodontal diseases are also a major affliction to mankind. Gingivitis, inflammation of gingival (gum) tissue, and periodontitis, inflammation and progressive loss of ligament and alveolar (socket) bone support to teeth are caused by bacteria which colonize tooth surfaces and occupy the gingival crevice area.

Routine daily prevention or removal of plaque by the patient is a cornerstone of dental therapy. Toothbrushes, dental floss and various other oral hygiene instruments can be used. These devices require motor skill and dexterity. The daily routines for adequate plaque removal require diligence, motivation, education and skill by the patient. These methods are often limited in their effectiveness in plaque removal. Conventional dental therapy has emphasized the restoration of dental caries with filling materials and crowns and replacement of lost teeth with prosthetic materials (e.g., implants, fixed bridges, partial and full dentures and dentures fixed to implants). These materials are all prone to dental plaque accumulation as well. Recurrent dental caries can occur at the margins of the natural tooth and the dental restoration or crown.

Often, accumulation of plaque at restorations and crowns near or below the gumline can exacerbate periodontal diseases. Periodontopathic microbes have been shown to inhabit the internal surfaces of the implant-abutment interface of two-stage dental implants in partially edentulous patients. The microbes colonize these surfaces within twenty-five days following the second stage surgery and placement of the healing abutment. The translocation of periodontopathic bacteria from residual dentition or secondary oral reservoirs (e.g., dorsal tongue surfaces and peri-tonsillar areas) may contribute to dental implant failure. See D. P. Callan, et al, DNA probe identification of bacteria colonizing internal surfaces of the implant-abutment interface: a preliminary study, J. Periodontol 76, 115-120 (2005). Growth of yeast organisms on removable partial or full dentures can result in oral candadiasis. Accumulation of dental plaque around orthodontic bands and brackets can lead to “white spot” lesions and dental caries.

The use of antimicrobial materials as dental materials would have a beneficial effect on the inhibition of recurrent disease or infection from colonization by oral microorganisms. These materials could provide continuous antimicrobial activity to protect the integrity of the restoration or prosthesis and inhibit disease by inhibiting colonization of microorganisms on the material surface.

Therefore, there remains a need for antimicrobial materials in dental materials, restorations and prostheses.

### SUMMARY OF THE INVENTION

The present invention relates to antimicrobial molecular entities for use in dental materials as restorative, prosthetic or adjunct agents for the prevention and inhibition of oral and dental diseases caused by oral microorganisms.

Accordingly, an embodiment of the present invention includes incorporation of an antimicrobial agent into the dental material so that the dental material exhibits antimicrobial activity.

In another embodiment of the present invention, an antimicrobial agent is placed as a surface coating which bonds to the dental material to provide persistent antimicrobial activity.

In yet another embodiment, an antimicrobial agent is incorporated into varnish or dental cement.

In still another embodiment of the present invention, the antimicrobial agent is in the form of a small molecule, oligomer, polymer or nanoparticle.

In another embodiment, the antimicrobial dental material is incorporated in or in the form of a dental restoration, dental prosthesis, dental crown, fixed bridge, removable partial denture, removable full denture, fixed full denture, dental implant, sealant, varnish, dental cement, orthodontic appliance or endodontic material.

These and various other advantages and features of novelty which characterize the invention are pointed out with particularity in the claims annexed hereto and forming a part hereof. However, for a better understanding of the invention, its advantages and objectives obtained by its use, reference should be made to the drawings which form a further part hereof, and to the accompanying descriptive matter in which there is illustrated and described preferred embodiments of the invention.

### BRIEF DESCRIPTION OF THE DRAWINGS

In the drawings, in which like reference numerals and letters indicate corresponding parts throughout the several views:

FIG. 1 is a diagrammatic view of a dental implant positioned in alveolar bone;

FIG. 2 is a diagrammatic view of a crown and bridge prosthesis ready for permanent cementing onto prepared abutment teeth;

FIGS. 3A through 3C are diagrammatic views of a carious lesion and its restoration. A carious lesion is illustrated in FIG. 3A. Caries removal with a dental bur is illustrated in FIG. 3B. A cross-sectional view of the restored tooth is illustrated in FIG. 3C;

FIGS. 4A through 4C are diagrammatic views of caries restoration and dental sealant application on a molar tooth. Dental caries in a pit and fissure occlusal defect is illustrated in cross-sectional view in FIG. 4A. Restoration of the carious lesion with a dental material and overlying dental sealant is illustrated in cross-sectional view in FIG. 4B and occlusal view in FIG. 4C;

FIGS. 5A and B are diagrammatic views of a dental veneer on a maxillary anterior tooth. FIG. 5A illustrates a frontal diagrammatic view of the dental veneer. A cross sectional diagrammatic view is illustrated in FIG. 5B; and

FIG. 6 is a diagrammatic view of a mandibular removable partial denture replacing missing posterior teeth.

### DETAILED DESCRIPTION OF THE INVENTION

Definitions

As used herein, the term “microorganism” refers to any noncellular or unicellular (including colonial) organism. Microorganisms include all prokaryotes. Microorganisms include bacteria, fungi, protozoa, and viruses. As used herein, the term “microbe” is synonymous with microorganism.

As used herein, the term “antimicrobial agent” includes a chemical entity that reduces the population of or that suppresses the multiplication or growth of a microbe or group or colony of microbes (e.g., a biofilm). An antimicrobial agent can effect microbial cell damage that is lethal and irreversible and results in complete microbial cell destruction or incapacitation. An antimicrobial agent can effect microbial cell damage that is reversible, such that if the organism is rendered free of the agent, it can again multiply.

Successful treatment with an antimicrobial agent can reduce or eliminate the symptoms of oral infection. Successful treatment with an antimicrobial agent can reduce the population of an oral microorganism of interest to a level at which it causes reduced, insignificant, or, even, no symptoms. Successful treatment with an antimicrobial agent can stop or reduce the formation or growth of a biofilm or reduce the rate of biofilm formation or growth.

Successful treatment can include reducing the population of microbe detected at a site in the oral cavity by at least about 50%. More successful treatment can include reducing the population of microbe detected at a site in the oral cavity by greater than a 90% reduction (1-log order reduction). More successful treatment can include reducing the population of microbe detected at a site in the oral cavity by greater than a 99% reduction (2-log order reduction). More successful treatment can include reducing the population of microbe detected at a site in the oral cavity by greater than a 99.99% reduction (4-log order reduction). More successful treatment can include reducing the population of microbe detected at a site in the oral cavity by greater than a 99.999% reduction (5-log order reduction) in such population

The Present Dental Materials and Methods Employing Them

The present invention relates to compositions and methods for utilization of antimicrobial dental materials in the restoration and prevention of oral infections. The antimicrobial agents include one or more molecular entities forming or incorporated into the dental material. The antimicrobial agents can be small molecules, oligomers, polymers, or a combination thereof.

In an embodiment, the antimicrobial agents can include the matrix of the dental material. In another embodiment, the antimicrobial agents can be combined or configured into the dental material.

In yet another embodiment, the antimicrobial agents can be a surface coating or component thereof bonded onto the dental material.

In another embodiment, the antimicrobial agents can be incorporated into the dental materials or surface coating in a homogenous manner. In still another embodiment, the antimicrobial agents can be incorporated into the dental materials or surface coating as a plurality of discrete units such as nanoparticles, microparticles or fibers.

Bacteria employ a cell-cell signaling mechanism to produce biofilms. For example, it is generally believed that all enteric bacteria and gram negative bacteria are capable of cell density regulation using acylated homoserine lactones (AHLs) as antoinducer molecules. Antimicrobial agents that can inhibit AHLs would be beneficial as antimicrobial agents. See S. Srinivasan, et al, Extracellular signal molecule(s) involved in the carbon starvation response of marine vibrio sp. Strain S14, J. Bacteriol 180, 201 (1988). In an embodiment, certain furanones are employed as antagonists of AHLs thus demonstrating antimicrobial activity.

In an embodiment, the method employs an antimicrobial agent that is a furanone or furanone derivative. Suitable furanones are disclosed in U.S. Pat. Nos. 6,337,347 and 6,455,031, the disclosures of which are incorporated by reference. Such furanones include compounds of Formula 1 or Formula 2: 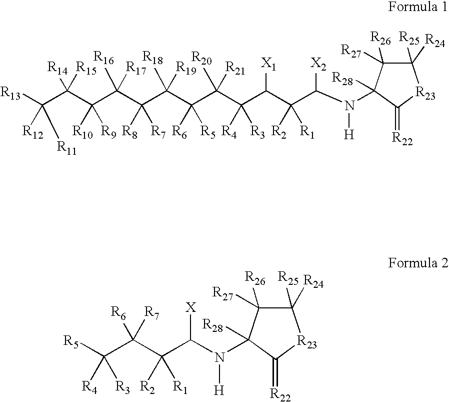
In Formulas 1 or 2 R₁-R₂₁ can independently be H, C₁-C₄ alkyl group (preferably CH₃), OH, NH₂, SH, or halogen (e.g., F, Cl, Br, or I); R₂₂ can independently be H, S, O and N (e.g., NR or NH), preferably S or O; R₂₄-R₂₈ can independently be H or halogen; and X, X₁, and X₂ can independently be O, S, H₂, or any combination of H plus one halogen or two halogens when one or more R groups is substituted. The furanone can be an optically active isomer.

In an embodiment, the furanone has Formula 1. In an embodiment of Formula 1, at least one of R₁-R₂₁ is halogen, or the alkylene chain of the molecule contains a sulfur in the chain.

In an embodiment of Formula 1, R₂₄-R₂₈ are H or halogen, and R₂₂-R₂₃ are H. In an embodiment of Formula 1, one or more carbons forming the backbone of the molecule are substituted with S or S-substituted moieties. In an embodiment of Formula 1, X₁ and/or X₂ is H₂, H plus halogen or two halogens. In an embodiment of Formula 1, R₂₂ is H, S, O or NH and R₂₃ is S, O or N. In an embodiment of Formula 1, the alkylene side chain contains one or more double bonds or triple bonds between carbon atoms within the alkylene side chain. In an embodiment of Formula 1, X₁-X₂ is H₂; H plus a halogen; two halogens; H plus OH or NH₂; or a double bonded O, NH, or S.

In an embodiment, the furanone has Formula 2. In an embodiment of Formula 2, at least one or R₁-R₇ is halogen, or the alkylene chain of the molecule contains a sulfur in the chain. In an embodiment of Formula 2, R₂₂ is H, S, O or NH and R₂₃ is S, O, or N. In an embodiment of Formula 2, the alkylene side chain contains one or more double bonds or triple bonds between carbon atoms within the alkylene side chain. In an embodiment of Formula 2, X is H₂; H plus a halogen; two halogens; H plus OH or NH₂; or a double bonded O, NH, or S.

The furanones of Formulas 1 or 2 can also include the above structures with modifications such as: 1) Alteration of the acyl side chain by increasing or decreasing its length. 2) Alteration of the structure of the acyl side chain, such as addition of a double bond or a triple bond between carbon atoms within the acyl side chain. 3) Substitution on carbons in the acyl side chain, e.g., the addition of a methyl group or other group such as an oxo-group, a hydroxyl group, an amino group, a sulfur atom, a halogen or dihalogen or some other atom or R-group to any location along the acyl side chain. 4) Substitution of carbons including the backbone of the acyl side chain with S or S substituted moieties or with N or N substituted moieties. 5) Substitution on the homoserine lactone ring portion of the molecule. For example: addition of a sulfur group to produce a thiolactone. 6) Halogenated acyl furanones have been shown to act as blockers to homoserine lactone cognate receptor proteins. 7) Ring size of the acyl side chain varying heterocylic moiety is variable. For example, 4-membered and 6-membered rings containing nitrogen (i.e., beta and delta lactams) are included.

The furanones of Formulas 1 and 2 include compounds such as compounds 1-12:

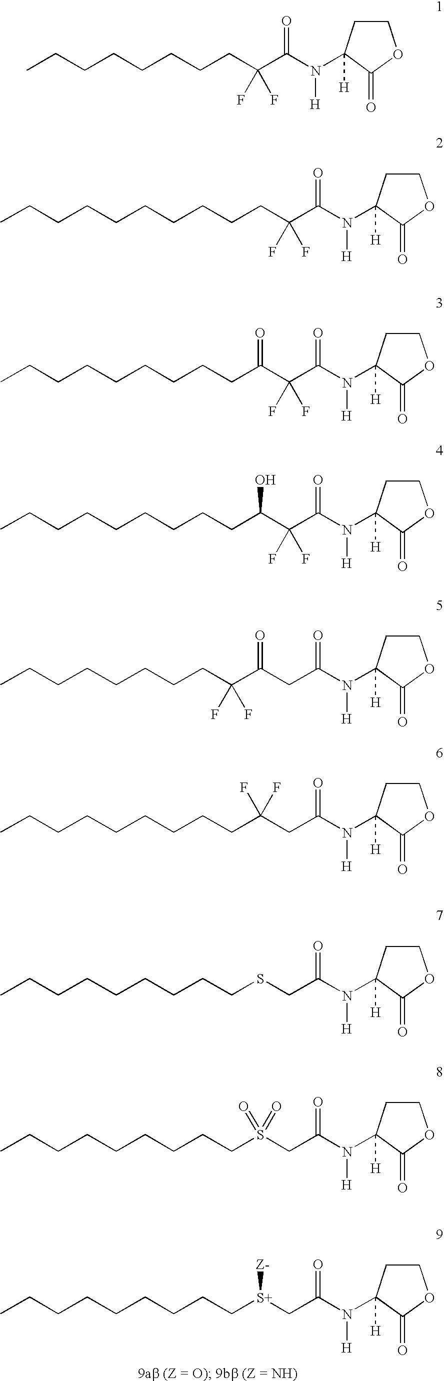

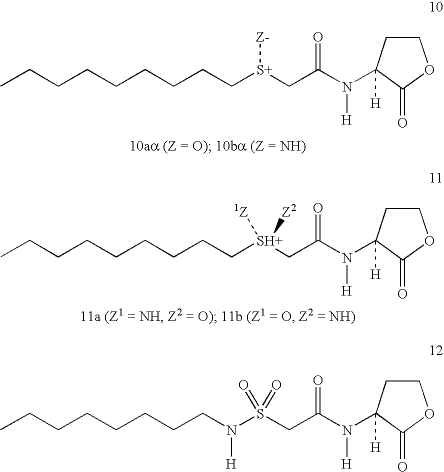

Suitable furanones are disclosed in U.S. Pat. Nos. 6,060,046 and 6,555,356, the disclosures of which are incorporated by reference. Such furanones include compounds of Formula 3:

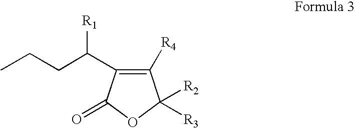

In Formula 3, R₁, R₂, and R₃ can independently be hydrogen, hydroxyl, alkyl containing from 1 to 10 carbon atoms, ether containing from 1 to 10 carbon atoms, ester containing from 1 to 10 carbon atoms, or halogenated alkene containing from 1 to 10 carbon atoms; or R₂ and R₃ together can include an unsubstituted or halogenated alkene containing from 1 to 10 carbon atoms and R4 can be hydrogen or halogen.

In an embodiment of Formula 3, R₁ is hydrogen, hydroxy or acetoxy; and R₂ and R₃ are independently single unsubstituted or halogenated methylene group. In an embodiment of Formula 3, R₁ is hydrogen, hydroxyl, ester or ether; and R₂ and R₃ are each together unsubstituted or halogenated methylene group. In an embodiment of Formula 3, R₂ is hydrogen or bromine, R₃ is halogen, and R₄ is hydrogen or bromine. In an embodiment of Formula 3, R₁ is hydrogen, hydroxyl, an ester or an ether group, and R₄ is bromine. In an embodiment of Formula 3, R₁ is hydrogen, hydroxy or acetoxy. In an embodiment of Formula 3, R₃ is chlorine, bromine or iodine. In an embodiment of Formula 3, R₁ is an acetyl group. In an embodiment of Formula 3, R₁ is a hydroxy group and R₂ and R₃ are each bromine.

In an embodiment, the furanone has Formula 4:

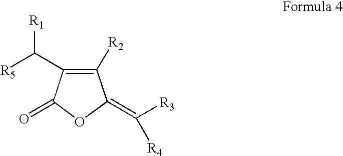

In Formula 4, R₁ is hydrogen, hydroxyl, acetoxy, ester or ether; R₂ is Br or H; R₃ and R₄ are independently hydrogen or halogen; and R₅ is C₁, C₃, C₅ or C₁₁ alkyl.

In an embodiment of Formula 4, R₁ is H, R₂ is Br, R₃ is Br, R₄ is Br, and R₅ is C₃ alkyl. In an embodiment of Formula 4, R₁ is H, R₂is Br, R₃ is H, R₄ is Br and R₅ is C₃ alkyl. In an embodiment of Formula 4, R is OAc, R₂ is Br, R₃ is H, R₄ is Br, and R₅ is C₃ alkyl. In an embodiment of Formula 4, R₁ is OH, R₂ is Br, R₃ is H, R₄ is Br, and R₅ is C₃ alkyl. In an embodiment of Formula 4, R₁ is OAc, R₂ is Br, R₃ is H, R₄ is I and R₅ is C₃ alkyl. In an embodiment of Formula 4, R₁ is H, R₂ is H, R₃ is Br, R₄ is Br, and R₅ is C₃ alkyl. In an embodiment of Formula 4, R₁ is OAc, R₂ is Br, R₃ is Br, R₄ is Br and R₅ is C₃ alkyl. In an embodiment of Formula 4, R₁ is H, R₂ is Br, R₃ is Br, R₄ is Br and R₅ is C₁ alkyl. In an embodiment of Formula 4, R₁ is H, R₂ is Br, R₃ is H, R₄ is Br, R₅ is C₁ alkyl. In an embodiment of Formula 4, R₁ is H, R₂ is H, R₃ is Br, R₄ is Br, and R₅ is C₁ alkyl. In an embodiment of Formula 4, R₁ is H, R₂ is Br, R₃ is H, R₄ is Br and R₅ is C₅ alkyl. In an embodiment of Formula 4, R₁ is H, R₂ is H, R₃ is Br, R₄ is Br, and R₅ is C₅ alkyl. In an embodiment of Formula 4, R₁ is H, R₂ is H, R₃ is Br, R₄ is Br, and R₅ is C₁₁ alkyl.

In an embodiment, the furanone has Formula 5: 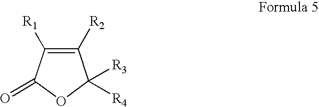
In formula 5, R₁, R₂, R₃, and R₄ are each independently hydrogen, halogen, hydroxyl, methyl, alkyl, ether or ester.

Production of another novel signaling molecule is also regulated by changes in environmental conditions associated with a shift from a planktonic, free-living condition to a colonizing or pathogenic existence in a host organism. This signaling molecule is termed autoinducer-2. See B. L. Brassler, et al, U.S. patent application Ser. No. 20020107364. In an embodiment, agents inhibiting autoinducer-2 are utilized as antimicrobial agents.

Biomimetic antimicrobial polymers and oligomers that mimic biologic properties of proteins can have antimicrobial properties. Non-protein mimetics of host defense proteins can exhibit potent and broad spectrum antimicrobial activity. Following the initial discovery of cecropins and magainins, antimicrobial peptides have become a large and growing class. See M. Zasloff, Antibiotic peptides as mediators of innate immunity, Curr Opin Immunol, 4, 3-7 (1992). Among the most well-studied antimicrobial peptides are the cationic amphiphillic alpha-helices, including the cecropins, magainins and melittin. See A. Tossi, et al, Amphipathic, alpha-helical and antimicrobial peptides, Biopolymers, 55, 4-30 (2000). Anti-bacterial activity of these agents is via bacterial cell wall lysis. Non-peptide oligomers can be synthesized to mimic this antimicrobial activity. See G. N. Tew, et al, De novo design of biomimetic antimicrobial polymers, PNAS, 99, 8, 5110-5114 (April, 2002). See also, W. F. DeGrado, et al, U.S. patent application Ser. No. 20040185257. Present examples include arylamides, hydrazides, calixarenes, diamines, nylomers, salicylamides, oxalamides, phenyl alkynyls, acrylamides, pyridines, mixtures thereof, or combinations thereof. In an embodiment of the present invention, non-peptide biomimetic small molecules, oligomers and polymers are utilized as antimicrobial agents.

In an embodiment, combinations of various antimicrobial agents can be incorporated into the dental materials.

In an embodiment, the antimicrobial agents have antimicrobial activity not dependant upon release activity. In an embodiment the dental material product can include an anti-bacterial agent, anti-fungal agent, or anti-viral agent.

In an embodiment, the dental material product can be a dental restoration, dental prosthesis, dental crown, fixed bridge, removable partial denture, removable full denture, fixed full denture, dental implant, sealant, varnish, dental cement, orthodontic appliance or endodontic material.

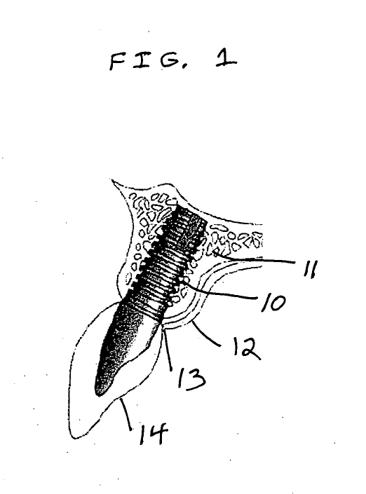

Referring now to FIG. 1, wherein a dental implant 10 is diagrammatically illustrated, the dental implant 10 is integrated to alveolar bone 11. The dental implant 10 exits the alveolar bone 11 into the oral cavity through the gingiva 12. The biomimetic antimicrobial molecular entities are placed at the gingival collar region 13 of the dental implant 10 to counteract the adverse effects of bacterial accumulation in this region, where the crown 14 meets the dental implant 10.

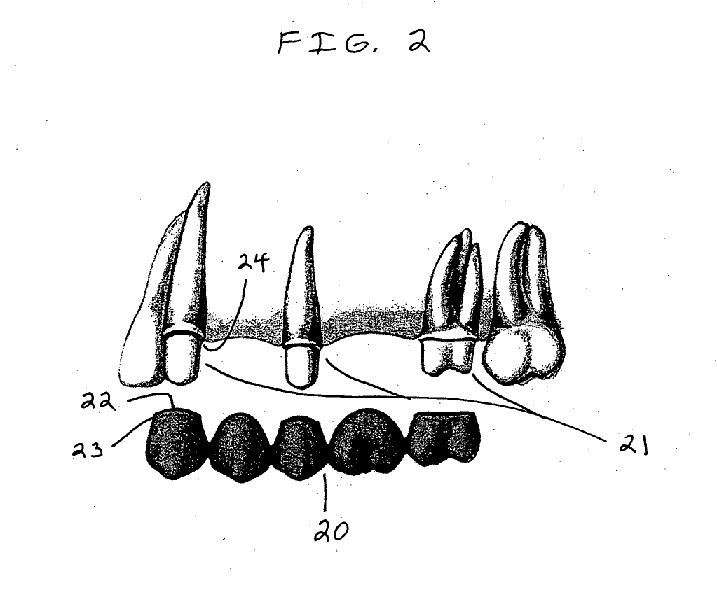

FIG. 2 illustrates a crown and bridge prosthesis 20 ready for cementation onto the abutment teeth 21. Dental cement 22 which contains a biomimetic antimicrobial agent is positioned inside the crown and bridge 20 abutments prior to placement of the prosthesis. Biomimetic antimicrobial molecular entities are also positioned in a surface coating at the external crown margin area 23 to act against bacterial accumulation in the gingival crevice 24.

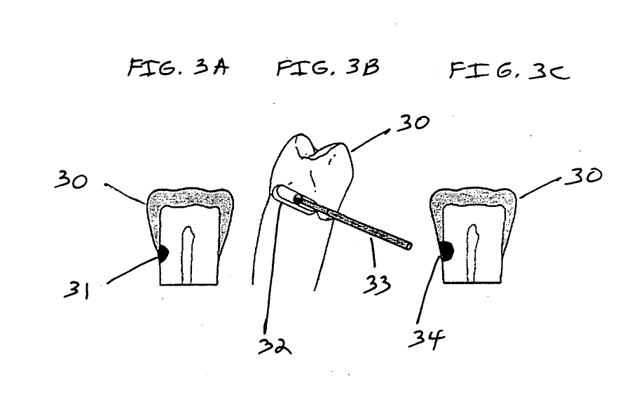

FIGS. 3A through 3C illustrate restoration of a carious lesion with a dental composite restoration. FIG. 3A illustrates a carious lesion 31 along the tooth 30 surface. FIG. 3B illustrates a cavity preparation 32 utilizing a dental bur 33. FIG. 3C illustrates a dental composite restoration 34 placed to restore the carious lesion 31 utilizing biomimetic antimicrobial molecular entities incorporated into the dental composite restoration as nanoparticles or a surface coating.

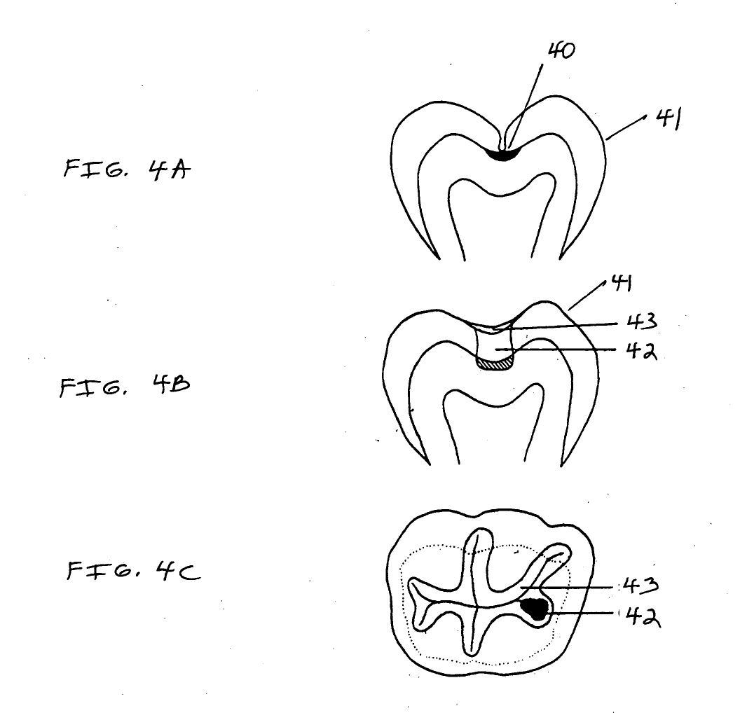

FIGS. 4A through 4C illustrate restoration of pit and fissure caries with a dental composite restoration and a dental sealant overlay. FIG. 4A illustrates a cross sectional view of a pit and fissure caries 40 in a molar tooth 41. FIG. 4B shows a cross sectional view of the cavity preparation restored with composite 42 and an overlying dental sealant 43. FIG. 4C illustrates an occlusal view of these restorations with the sealant 43 containing biomimetic antimicrobial molecular entities.

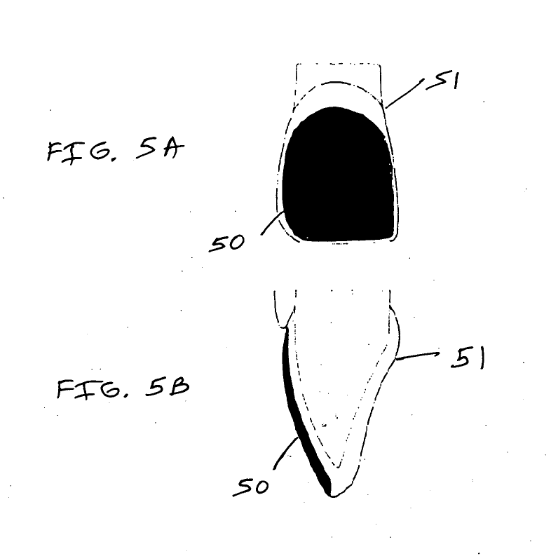

FIGS. 5A and 5B illustrate diagrammatically a dental veneer 50 on a maxillary central incisor 51. The dental veneer 50 is overcoated with biomimetic antimicrobial molecular entities. FIG. 5A illustrates diagrammatically a labial (frontal) view. FIG. 5B shows diagrammatically a cross-sectional view.

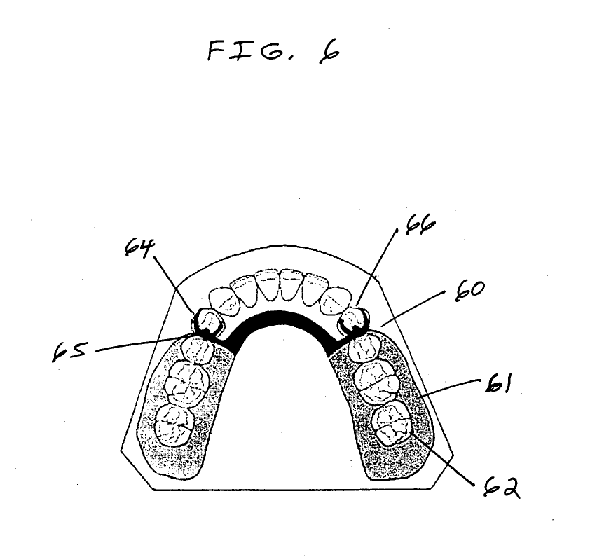

FIG. 6 illustrates diagrammatically a mandibular removable partial denture 60 replacing missing posterior teeth with an acrylic denture base 61 and embedded denture teeth 62. A metal lingual bar 63 connects the two posterior dental base 61 components of the removable partial denture 60. A metal retentive arm 64 and occlusal rest 65 fasten the removable partial denture 60 to a tooth 66 on both sides giving stability to the function and fit of the removable partial denture 60. Biomimetic antimicrobial molecular entities are placed as an overcoat on the metal portions and as nanoparticles in the denture base 61 or as a surface coating on the dental base 61 portions of the removable partial denture.

The dental materials of the invention contain effective amounts of antimicrobial agents. An “effective amount” of an antimicrobial agent is an amount sufficient to kill microorganisms on a surface of the dental material, or to prevent or inhibit their growth. The effective amount of an antimicrobial agent will vary depending upon, for example, the specific agent, the type of dental material and its composition, and other variables that may affect the operating conditions of the dental material such as temperature and moisture content of the mouth.

The determination of an effective amount of a selected antimicrobial agent for use in a selected dental material is within the ability of one of skill in the art. By way of nonlimiting example, the antimicrobial agent may be present in an amount of about 25%-100% by weight of a coating that is applied to the surface of a dental material. In a further nonlimiting example, the antimicrobial agent may be present in an amount of about 0.1% to about 10% by weight of a resin or polymer that is molded into a dental material. In another nonlimiting example, the antimicrobial agent may include a polymer that is molded into a dental material.

Dental restorations, prostheses, crowns, bridges, dentures, implants, sealants, varnishes, cements, orthodontic appliances, and endodontic materials are commonly formed from structural components including polymers, metals, or ceramics. Methods for making dental materials from these structural components are known, and will vary according to the nature of the structural component. Accordingly, methods for making the antimicrobial-containing dental materials of the invention will also vary according to the structural components used to form a dental material, as well as according to the manner in which an antimicrobial agent is to be associated with the dental material, for example as a coating or a component of a coating that is applied to the dental material, as an agent incorporated into the dental material, or by forming the matrix of the dental material.

In embodiments where the antimicrobial agent is a coating material or a component of a coating material, the coating material is selected to be compatible both with the antimicrobial agent and the dental material to which the coating will be applied. The coating material may be, for example, a liquid, a gel, or a paste. In an embodiment, the coating material includes or is a varnish. In an embodiment, the coating includes or is a cement. In an embodiment, the coating material includes or is a sealant. In these embodiments, the coating material may include other ingredients that are conventionally present in such coatings, in addition to the antimicrobial agent. The coating material may include an orally acceptable bonding material known in the art, such as an adhesive, to bond the coating to the dental material. In some embodiments, the coating material itself is an antimicrobial agent-containing dental material of the invention for direct application to a tissue within the oral cavity.

The coating material may contain components intended to enhance the activity of the anti-microbial agent. In an embodiment, the coating material is intended to be released into the oral cavity over a period of time, and may contain an agent known to enhance delivery of antimicrobial agents, such as the commercially available copolymer methylvinyl ether-maleic anhydride. The coating material may also contain a known orally acceptable surface-active agent such as an anionic, nonionic, or ampholytic surfactant, to aid in the dispersion of an antimicrobial agent into the oral cavity.

Coatings containing antimicrobial agents can be applied to a wide variety of materials to form an antimicrobial-containing dental material of the invention. In an embodiment, the antimicrobial agent is incorporated into a coating material by thoroughly mixing the agent with the other ingredients of the coating to produce an antimicrobial agent-containing coating material. Alternatively, a coating material may be produced according to conventional methods, and the antimicrobial agent then mixed thoroughly with the coating material until a uniform mixture is obtained. In an embodiment, the antimicrobial agent is incorporated into nanoparticles, microparticles, or fibers according to methods known in the art, and the particles or fibers mixed with the coating material, or mixed with the other ingredients that form the coating material, until homogeneously distributed to form an antimicrobial agent-containing coating material.

The antimicrobial agent-containing coating material can be applied to a surface of a dental material using any known technique, such as by painting, spraying, or dipping. In an embodiment, the coating is applied to a dental material using a powder coating process. In an embodiment, the antimicrobial agent is blended with a powder, such as polymer powders known to be orally acceptable, to form a composite. The composite is grounded or melt atomized to produce a powder that is applied to a surface of, for example, a metallic dental material, using conventional powder coating processes.

Alternatively, a dispersion of a polymer powder and the antimicrobial agent can be formed. In an embodiment, the dispersion contains a binder to coat the anti-microbial agent onto the polymer powder particles. The liquid is then evaporated, and the powder applied to a dental material using conventional powder coating processes, and the dental material then dried and baked to bond the coating containing the antimicrobial agent to the dental material. In another embodiment, a powder is coated onto the surface of the dental material according to conventional methods, and a liquid dispersion of the anti-microbial agent is coated onto the powder coating. The dental material is then dried and baked to incorporate the anti-microbial agent into the coating.

In an embodiment, the antimicrobial agent-containing coating is a dental varnish, and is painted onto a dental material and allowed to dry into a film. In an embodiment, a sealing layer is applied over the film to sustain antimicrobial activity for a longer length of time. In an embodiment, the sealing layer includes or is a solvated polyurethane that can be cured by evaporation of the solvent.

In addition to applying antimicrobial agents to a surface of a dental material in the form of a coating, antimicrobial agents can be incorporated into polymeric materials commonly used in the art to form a wide variety of antimicrobial dental appliances of the invention. For example, the dental material may be formed from an acrylic polymer, a silicone rubber, a hydrophilic polymer, an elastomer such as a natural or synthetic rubber, a urethane, polypropylene, silicon, polystyrene, polyethylene, polyvinyl chloride, polycarbonates, thermoplastics, ethylene vinyl acetate, or polyester resins such as polycaprolactone, or mixtures thereof.

The incorporation of an antimicrobial agent into a polymeric material generally involves blending the agent with the polymer prior to the further processing steps necessary to produce the dental material. In the blending step, the anti-microbial agent is compounded with the polymeric material to form a blended composition using conventional equipment, such as a high speed mixer, or using a mill having several rollers. By encapsulating the antimicrobial agent into the polymer, the agent can survive the high temperatures that may be experienced when, for example, the polymer is injection molded or extruded using conventional methods to shape the polymer into the desired form.

In an embodiment, the antimicrobial agent is first mixed with a low density polymer such as polypropylene, polystyrene, or polyethylene, to generate a batch of pellets that can more easily be mixed with polymeric materials during the blending step described above.

In an embodiment, the antimicrobial agent is blended as described above, but with polymer precursors instead of polymers. The polymer precursors are then polymerized in situ within the oral cavity using a polymerization initiator according to known techniques. Orally acceptable polymer precursors such as alkyl methacrylates having an oxyphosphorus group, are well known in the art and are used for the generation of a variety of dental materials such as dental adhesives, pulp caps, dental liners, and other dental restoratives.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

All publications and patent applications in this specification are indicative of the level of ordinary skill in the art to which this invention pertains.

It is to be understood, however, that even though numerous characteristics and advantages of the invention have been set forth in the foregoing description, together with details of the structure and function of the invention, the disclosure is illustrative only, and changes may be made in detail, especially in matters of shape, size and arrangement of parts within the principle of the invention, to the full extent indicated by the broad general meaning of the terms in which the appended claims are expressed.

We claim:

1. A method of treating an oral disease, comprising:
applying to the oral cavity of a subject at risk of oral microbial infection a dental material comprising an antimicrobial agent effective for inhibiting an oral microorganism.

2. A method according to claim 1, wherein the oral disease comprises dental caries, oral candadiasis, oral herpes, periodontal disease, or periimplantitis.

3. A method according to claim 1, wherein the antimicrobial agent comprises a small molecule, an oligomer, a polymer, or a nanoparticle.

4. A method according to claim 1, wherein the antimicrobial agent is incorporated into the dental material.

5. A method according to claim 1, wherein the antimicrobial agent is a coating on the exterior of the dental material.

6. A method according to claim 1, wherein the antimicrobial agent comprises a non-protein mimetic of a host defense protein, a biofilm disrupter, or a combination thereof.

7. A method according to claim 6, wherein the non-protein mimetic comprises arylamides, hydrazides, calixarenes, diamines, nylomers, salicylamides, oxalamides, phenyl alkynls, acrylamides, pyridines, or a mixture thereof.

8. A method according to claim 6, wherein the biofilm disrupter is an antagonist of acylated homoserine lactones.

9. A method according to claim 8, wherein the biofilm disrupter comprises a furanone biofilm disrupter.

10. A method according to claim 9, wherein the furanone biofilm disrupter has Formula 1, 2, 3, 4, or 5: 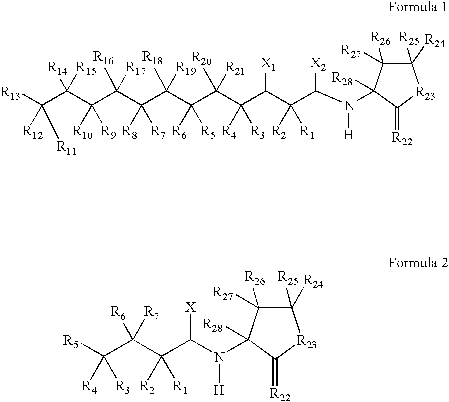
wherein: R₁-R₂₁ are independently H, C₁-C₄ alkyl group, OH, NH₂, SH, or halogen; R₂₂ and R₂₃ are independently H, S, O, NR, or NH; R₂₄-R₂₈ are independently H or halogen; and X, X₁, and X₂ are independently O, S, H₂, or any combination of H plus one halogen or two halogens when one or more R groups is substituted; 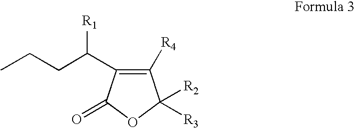
wherein, R₁, R₂ and R₃ are independently hydrogen, hydroxyl, alkyl containing from 1 to 10 carbon atoms, ether containing from 1 to 10 carbon atoms, ester containing from 1 to 10 carbon atoms, or halogenated alkene containing from 1 to 10 carbon atoms; or R₂ and R₃ together are unsubstituted or halogenated alkene containing from 1 to 10 carbon atoms and R₄ is hydrogen or halogen; 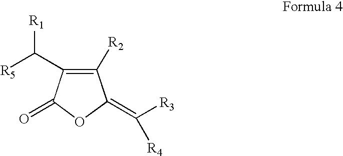
wherein, R₁ is hydrogen, hydroxyl, acetoxy, ester or ether; R₂ is Br or H; R₃ and R4 are independently hydrogen or halogen; and R₅ is C₁, C₃, C₅, or C₁₁ alkyl; or 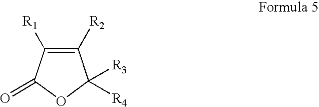
wherein, R₁, R₂, R₃ and R₄ are each independently hydrogen, halogen, hydroxyl, methyl, alkyl, ether or ester.

11. A method according to claim 6, wherein the biofilm disrupter is an inhibitor of autoinducer-2.

12. A method according to claim 6, wherein the biofilm disrupter is an agent that inhibits or disrupts the biofilm extracellular matrix.

13. A method according to claim 1, wherein the dental material is a dental cement, luting agent, dental base, dental liner, dental (cavity-liner) varnish, or combination thereof.

14. A method according to claim 13, wherein the preventive varnish, dental sealant or dental coating erodes or degrades to release the antimicrobial agent in a controlled, sustained manner.

15. A method according to claim 1, wherein the dental material is a dental restoration, dental prosthetic restoration, crown prosthesis, bridge prosthesis, removable partial denture, removable full denture, dental implant, dental implant component, fixed partial denture, fixed full denture, endodontic material, orthodontic appliance, band, bracket, space maintainer, preventive varnish, dental sealant, dental coating, or combination thereof.

16. A method according to claim 15, wherein the dental coating attaches to natural teeth or other dental materials.

17. A dental material comprising an antimicrobial agent;
wherein the dental material comprises:
dental cement, luting agent, dental base, dental liner, dental (cavity-liner) varnish, or combination thereof; or
dental restoration, dental prosthetic restoration, crown prosthesis, bridge prosthesis, removable partial denture, removable full denture, dental implant, dental implant component, fixed partial denture, fixed full denture, endodontic material, orthodontic appliance, band, bracket, space maintainer, preventive varnish, dental sealant, dental coating, or combination thereof.

18. The dental material of claim 17, wherein the antimicrobial agent comprises a non-protein mimetic of a host defense protein, a biofilm disrupter, or a combination thereof.

19. A method according to claim 18, wherein the non-protein mimetic comprises arylamides, hydrazides, calixarenes, diamines, nylomers, salicylamides, oxalamides, phenyl alkynls, acrylamides, pyridines, or a mixture thereof.

20. A method according to claim 18, wherein the biofilm disrupter is an antagonist of acylated homoserine lactones.

21. A method according to claim 18, wherein the biofilm disrupter comprises a furanone biofilm disrupter.

22. A method according to claim 21, wherein the furanone biofilm disrupter has Formula 1, 2, 3, 4, or 5: 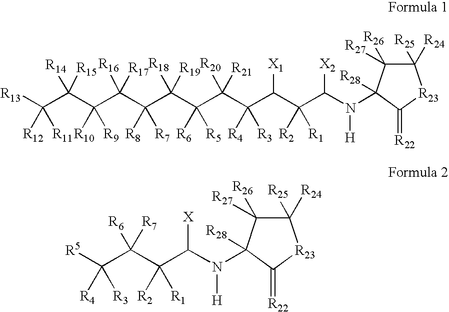
wherein: R₁-R₂₁ are independently H, C₁-C₄ alkyl group, OH, NH₂, SH, or halogen; R₂₂ and R₂₃ are independently H, S, O, NR, or NH; R₂₄-R₂₈ are independently H or halogen; and X, X₁, and X₂ are independently O, S, H₂, or any combination of H plus one halogen or two halogens when one or more R groups is substituted; 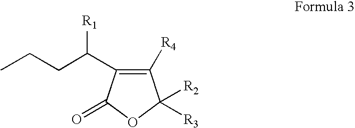
wherein, R₁, R₂ and R₃ are independently hydrogen, hydroxyl, alkyl containing from 1 to 10 carbon atoms, ether containing from 1 to 10 carbon atoms, ester containing from 1 to 10 carbon atoms, or halogenated alkene containing from 1 to 10 carbon atoms; or R₂ and R₃ together are unsubstituted or halogenated alkene containing from 1 to 10 carbon atoms and R₄ is hydrogen or halogen; 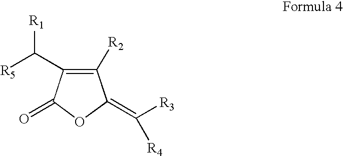
wherein R₁ is hydrogen, acetoxy, ester or ether; R₂ is Br or H; R₃ and R₄ are independently hydrogen or halogen; and R₅ is C₁, C₃, C₅, or C₁₁ alkyl; or 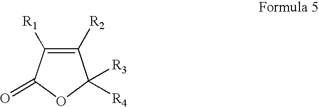
wherein, R₁, R₂, R₃ and R₄ are each independently hydrogen, halogen, hydroxyl, methyl, alkyl, ether or ester.

23. A method according to claim 18, wherein the biofilm disrupter is an inhibitor of autoinducer-2.

24. A method according to claim 18, wherein the biofilm disrupter is an agent that inhibits or disrupts the biofilm extracellular matrix.

25. A method according to claim 17, wherein the antimicrobial agent is incorporated into the dental material.

26. A method according to claim 17, wherein the antimicrobial agent is a coating on the exterior of the dental material.
